(19) United States
(12) Patent Application Publication
(10) Pub. No.: US 2016/0270677 A1
(43) Pub. Date: Sep. 22, 2016

(54) OPTICAL SENSING APPARATUS AND OPERATING METHOD THEREOF

(71) Applicant: uPI semiconductor corp., Zhubei City (TW)

(72) Inventor: YU-TING LIN, Zhubei City (TW)

(21) Appl. No.: 15/054,168

(22) Filed: Feb. 26, 2016

(30) Foreign Application Priority Data
Mar. 20, 2015 (TW) 104109027

(51) Int. Cl. A61B 5/024; A61B 5/00; G01D 5/34

(52) U.S. Cl. CPC A61B 5/02427; G01D 5/34; A61B 5/7221; A61B 5/7207; A61B 2562/0257

(57) ABSTRACT

An optical sensing apparatus includes a sensing unit, an analog/digital conversion unit, a switching unit, a first processing unit, and a second processing unit. The sensing unit provides a light sensing signal. The analog/digital conversion unit is coupled to sensing unit and converts light sensing signal into a digital light sensing signal. The switching unit is coupled to analog/digital conversion unit and selectively switched to a first mode or a second mode according to a mode control signal. The first processing unit and second processing unit are coupled to analog/digital conversion unit and switching unit. When switching unit is switched to first mode according to mode control signal, first processing unit generates a photoplethysmography (PPG) signal according to digital light sensing signal; when switching unit is switched to second mode according to mode control signal, second processing unit generates a proximity sensing signal according to digital light sensing signal.

### BACKGROUND OF THE INVENTION

1. Field of the Invention

The invention relates to optical sensing; in particular, to an optical sensing apparatus having both proximity sensing function and heart rate sensing function and operating method thereof.

2. Description of the Prior Art

In general, the heme in the blood absorbs lights and the pulses formed by arterial rhythm cause the change of the artery thickness and the variation of intravascular blood levels; therefore, the conventional optical sensor can use a photoplethysmoggraphy (PPG) technology to sense the heart rate. Its principles includes using a light source to emit lights to penetrate through the skin and enter into the artery and dispose a penetrating light sensor at opposite side to sense the intensities of the penetrating lights or dispose a reflected light sensor at the same side to sense the intensities of the reflected lights. The intensities of the penetrating lights or reflected lights sensed by the light sensors are varied with the intravascular blood levels. The intensity variation of the penetrating lights or reflected lights is called photoplethysmoggraphy (PPG) and PPG can be used to show the variation of pulses. After continuously sensing the PPG signals for a period of time, the regularity of the variation of pulses can be detected to find pulse peak values and the heart rate can be obtained according to the time interval between two adjacent pulse peak values.

However, not only the pulses, but also other factors in the blood will cause the changes in light absorption of heme; for example, the factors of vessel wall, venous blood, skin, and external detector optical attenuation. And, the background value in the PPG signal formed by these factors is far larger than the dynamic changing value in the PPG signal formed by the pulses, even 50 times or 100 times. In addition, during the practical measurement, the contact gap between the optical sensor and skin is easily changed due to the body shaking, and the moving noises in the PPG signal will be formed accordingly. It is hard to maintain the PPG signal including the moving noises at the same base line and accurately determine the pulse peak values.

In order to reduce the above-mentioned moving noises, the conventional optical sensor usually uses the high-pass filter formed by analog circuits to reduce the background value in the PPG signal and the moving noises slower than the heart rate. However, since the filtering frequency of the high-pass filter is usually set about 0.5 Hz, on the circuit implementations, the capacitors having high capacitance must be used to form the high-pass filter. No matter the capacitors are disposed outside or inside the optical sensor, the volume and cost of the optical sensor will be increased and not conducive to be used in portable or wearable electronic devices. Furthermore, the range of moving noise variation that the high-pass filter can withstand also has its limitation and it fails to be dynamically adjusted in response to different moving noises.

### SUMMARY OF THE INVENTION

Therefore, the invention provides an optical sensing apparatus and operating method thereof to solve the above-mentioned problems occurred in the prior arts.

An embodiment of the invention is an optical sensing apparatus. In this embodiment, the optical sensing apparatus includes a sensing unit, an analog/digital conversion unit, a switching unit, a first processing unit, and a second processing unit. The sensing unit provides a light sensing signal. The analog/digital conversion unit is coupled to the sensing unit and converts the light sensing signal into a digital light sensing signal. The switching unit is coupled to the analog/digital conversion unit. The switching unit selectively switches to a first mode or a second mode according to a mode control signal. The first processing unit is coupled to the analog/digital conversion unit and the switching unit. The second processing unit is coupled to the analog/digital conversion unit and the switching unit. When the switching unit is switched to the first mode according to the mode control signal, the first processing unit generates a photoplethysmography (PPG) signal according to the digital light sensing signal; when the switching unit is switched to the second mode according to the mode control signal, the second processing unit generates a proximity sensing signal according to the digital light sensing signal.

In an embodiment, the first processing unit performs a first rolling average calculation and a second rolling average calculation on the digital light sensing signal via a first sample size and a second sample size respectively, and the second sample size is larger than the first sample size.

In an embodiment, the first sample size is 0.1˜0.2 times of a sampling time and the second sample size is 0.5˜1 times of the sampling time.

In an embodiment, after the first processing unit further obtains the digital light sensing signal without high-frequency noises and ultra-low frequency motion noises according to calculation results of the first rolling average calculation and the second rolling average calculation, the first processing unit detects a PPG peak value and generates the PPG signal according to a detection result of the PPG peak value.

In an embodiment, the first processing unit further determines whether to adjust a light source output or adjust a magnification of the light sensing signal according to the detection result of the PPG peak value.

In an embodiment, the first processing unit performs a rolling average calculation on the digital light sensing signal via a sample size and removes ultra-low frequency motion noises from the digital light sensing signal according to a calculation result of the rolling average calculation and removes high-frequency noises from the digital light sensing signal through low-pass filtering, and then the first processing unit detects a PPG peak value and generates the PPG signal according to a detection result of the PPG peak value.

In an embodiment, the first processing unit performs a rolling average calculation on the digital light sensing signal via a sample size and removes ultra-low frequency motion noises from the digital light sensing signal according to a calculation result of the rolling average calculation and removes high-frequency noises from the digital light sensing signal through low-pass filtering, and then the first processing unit detects a PPG peak value and generates the PPG signal according to a detection result of the PPG peak value.

In an embodiment, the sample size is 0.1˜0.2 times of a sampling time.

In an embodiment, the mode control signal is generated by an application program performed by the optical sensing apparatus.

In an embodiment, the mode control signal is generated when the optical sensing apparatus is continuously pressed for a default time.

Another embodiment of the invention is a method of operating an optical sensing apparatus. In this embodiment, the method includes: (a) the optical sensing apparatus converting the light sensing signal into a digital light sensing signal; (b) the optical sensing apparatus selectively switching to a first mode or a second mod according to a mode control signal; (c1) when the optical sensing apparatus switches to the first mode, the optical sensing apparatus generating a photoplethysmography (PPG) signal according to the digital light sensing signal; and (c2) when the optical sensing apparatus switches to the second mode, the optical sensing apparatus generating a proximity sensing signal according to the digital light sensing signal.

Compared to the prior arts, the optical sensing apparatus and operating method thereof in the invention will amplify the analog sensing signal of the optical sensor and convert it into the digital sensing signal through high-resolution analog/digital conversion, and then use digital way to reduce the background noises and moving noises and calculate the heart rate, and feedback the calculating result to determine whether to adjust the light source or the magnification of the sensing signal. The optical sensing apparatus and operating method thereof in the invention have following advantages:

(1) The optical sensing apparatus of the invention can have both the proximity sensing function and heart rate sensing function, and the light source and the corresponding optical sensor are disposed at the same side of the optical sensing apparatus, when the optical sensing apparatus contacts with the human body, the intensity of the emitting light can be adjusted to penetrate through the skin and enter into the artery, and then the optical sensor senses the intensity of the reflected lights and performs detection and calculation of the PPG signal in the reflected light sensor mode.

(2) The optical sensing apparatus of the invention uses the digital ways (the rolling average calculation method and low-pass filtering method) to remove the high-frequency background noises and ultra-low frequency moving noises from the digital light sensing signal; therefore, no high-pass filter including capacitors with high capacitance is necessary in the optical sensing apparatus to largely reduce the volume and cost of the optical sensing apparatus. In addition, since the optical sensing apparatus of the invention uses the digital calculation method (the rolling average calculation method) to average the original signals, it has adaptability to be dynamically adjusted in response to different moving noises.

(3) The optical sensing apparatus of the invention uses the analog/digital converter having high resolution to convert the analog light sensing signal into the digital light sensing signal to retain a high dynamic range in response to different background values under different usage scenarios. In addition, since the high-frequency background noises and ultra-low frequency moving noises are removed from the digital light sensing signal, the optical sensing apparatus of the invention only needs simple calculation structure to obtain the peak values of the PPG signal to calculate the heart rate, the circuit structure of the optical sensing apparatus of the invention can be simplified and the cost can be decreased.

(4) The optical sensing apparatus of the invention can be switched to operate the proximity sensing function or the heart rate sensing function according to practical needs. Since it is small and cheap, it can be widely used in smart phones, tablet PCs, notebook PCs and various kinds of wearable electronic devices and have great market potential.

The advantage and spirit of the invention may be understood by the following detailed descriptions together with the appended drawings.

### BRIEF DESCRIPTION OF THE APPENDED DRAWINGS

FIG. 1 illustrates a functional block diagram of the optical sensing apparatus in an embodiment of the invention.

FIG. 2 illustrates a functional block diagram of an embodiment of the first digital processing unit 14 of FIG. 1.

FIG. 3A illustrates a waveform schematic diagram of the first digital light sensing signal S1 outputted by the analog/digital conversion unit 12; FIG. 3B illustrates a waveform schematic diagram of the second digital light sensing signal S2 obtained by performing a first rolling average calculation on the first digital light sensing signal S1 via a first sample size; FIG. 3C illustrates a waveform schematic diagram of the third digital light sensing signal S3 obtained by performing a second rolling average calculation on the first digital light sensing signal S1 via a second sample size; FIG. 3D illustrates a waveform schematic diagram of the PPG signal S4 obtained by subtracting the third digital light sensing signal S3 from the second digital light sensing signal S2; FIG. 3E illustrates a waveform schematic diagram of the heart rate signal S5 obtained according to the peak values of the PPG signal S4.

FIG. 4A illustrates a waveform schematic diagram of the first digital light sensing signal S1′ outputted by the analog/digital conversion unit 12; FIG. 4B illustrates a waveform schematic diagram of the second digital light sensing signal S2′ obtained by performing a rolling average calculation on the first digital light sensing signal S1′ via a sample size; FIG. 4C illustrates a waveform schematic diagram of the PPG signal S3′ obtained by subtracting the first digital light sensing signal S1′ from the second digital light sensing signal S2′; FIG. 4D illustrates a waveform schematic diagram of the PPG signal S4′ obtained by performing low-pass filtering on the PPG signal S3′; FIG. 4E illustrates a waveform schematic diagram of the heart rate signal S5′ obtained according to the peak values of the PPG signal S4′.

FIG. 5 illustrates a flowchart of the optical sensing apparatus operating method in another embodiment of the invention.

FIG. 6 illustrates a flowchart of the optical sensing apparatus operating method in still another embodiment of the invention.

### DETAILED DESCRIPTION OF THE INVENTION

Exemplary embodiments of the present invention are referenced in detail now, and examples of the exemplary embodiments are illustrated in the drawings. Further, the same or similar reference numerals of the elements/components in the drawings and the detailed description of the invention are used on behalf of the same or similar parts.

In the following embodiments, if an element is “connected” or “coupled” to another element, the element may be directly connected or coupled to the another element, or there may be any elements or specific materials (e.g., colloid or solder) disposed between the element and the another element. The term “circuit” may be at least one element or multiple elements, or elements actively and/or passively coupled to provide proper function(s). The term “signal” may be at least one current, voltage, load, temperature, data or other signals.

A preferred embodiment of the invention is an optical sensing apparatus. In this embodiment, the optical sensing apparatus can have both the proximity sensing function and heart rate sensing function and it can be switched to operate the proximity sensing function or the heart rate sensing function according to practical needs. Since it is small and cheap, it can be widely used in smart phones, tablet PCs, notebook PCs and various kinds of wearable electronic devices, but not limited to this.

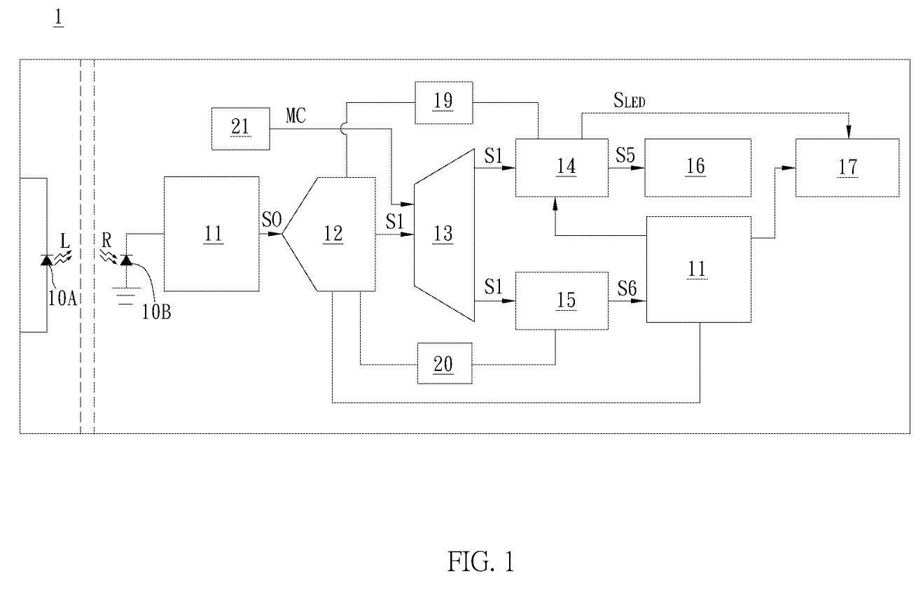

Please refer to FIG. 1. FIG. 1 illustrates a functional block diagram of the optical sensing apparatus in this embodiment. As shown in FIG. 1, the optical sensing apparatus 1 includes a light source unit 10A, a sensing unit 10B, an analog processing unit 11, an analog/digital conversion unit 12, a switching unit 13, a first digital processing unit 14, a second digital processing unit 15, a buffer unit 16, a driving unit 17, a transmission interface 18, a first oscillating unit 19, a second oscillating unit 20 and a mode control unit 21. Wherein, the light source unit 10A and the sensing unit 10B are disposed at the same side of the optical sensing apparatus 1; the sensing unit 10B is coupled between the ground terminal and the analog processing unit 11; the analog processing unit 11 is coupled to the analog/digital conversion unit 12; the analog/digital conversion unit 12 is coupled to the switching unit 13, the transmission interface 18, the first oscillating unit 19 and the second oscillating unit 20; the switching unit 13 is coupled to the first digital processing unit 14 and the second digital processing unit 15 respectively; the first digital processing unit 14 is coupled to the buffer unit 16, the driving unit 17, the transmission interface 18 and the first oscillating unit 19; the second digital processing unit 15 is coupled to the transmission interface 18 and the second oscillating unit 20; the buffer unit 16 is coupled to the transmission interface 18; the driving unit 17 is coupled to the transmission interface 18; the mode control unit 21 is coupled to the switching unit 13.

Next, the above-mentioned units of the optical sensing apparatus 1 will be introduced in detail as follows.

The light source unit 10A and the sensing unit 10B are both disposed at the same side of the optical sensing apparatus 1 and the sensing unit 10B corresponds to the light source unit 10A; therefore, the light source unit 10A and the sensing unit 10B can be used as “a reflective proximity sensor”. When the light source unit 10A emits an incident light L having a specific wavelength to a target and reflected by the target, the sensing unit 10B will receive a reflected light R having the specific wavelength reflected by the target, and then the analog processing unit 11 will perform analog processing procedures including the amplifying process on the reflected light R and output an analog light sensing signal S0 to the analog/digital conversion unit 12.

In fact, the light source unit 10A can be a light-emitting diode to emit the incident light L having the specific wavelength; the sensing unit 10B can be a light sensor corresponding to the light source unit 10A to receive the reflected light R having the specific wavelength, but not limited to this. In addition, the magnification that the analog processing unit 11 amplifies the reflected light R can be 1˜128, but not limited to this.

Then, the analog/digital conversion unit 12 will convert the analog light sensing signal S0 into a first digital light sensing signal S1 and output the first digital light sensing signal S1 to the switching unit 13.

It should be noticed that the analog/digital conversion unit 12 in this embodiment is an analog/digital converter having high resolution, so that it can retain a high dynamic range in response to different background values under different usage scenarios. In fact, the resolution of the analog/digital conversion unit 12 is at least 16 bits, but not limited to this.

In this embodiment, the switching unit 13 will receive a mode control signal MC from the mode control unit 21 and selectively switch to a first mode or a second mode according to the mode control signal MC. In fact, the first mode and the second mode can be a heart rate sensing mode and a proximity sensing mode respectively, but not limited to this.

It should be noticed that the mode control signal MC outputted by the mode control unit 21 can be generated by an application program performed by the optical sensing apparatus 1 or generated when the optical sensing apparatus 1 is continuously pressed for a default time without any specific limitations. For example, the user can select the first mode or the second mode performed by the optical sensing apparatus 1 through the application program, or the user can also select the first mode or the second mode performed by the optical sensing apparatus 1 through continuously pressing the optical sensing apparatus 1 for 3 seconds.

Next, the conditions that the switching unit 13 switches to the first mode and the second mode will be introduced in detail as follows.

(1) When the switching unit 13 switches to the first mode according to the mode control signal MC, the switching unit 13 transmits the first digital light sensing signal S1 to the first digital processing unit 14. The first oscillating unit 19 coupled to the first digital processing unit 14 is a high-speed oscillator used to provide a first clock signal to the first digital processing unit 14. In this embodiment, the first digital processing unit 14 is a digital signal processor used to generate a heart rate signal S5 according to the first digital light sensing signal S1, but not limited to this.

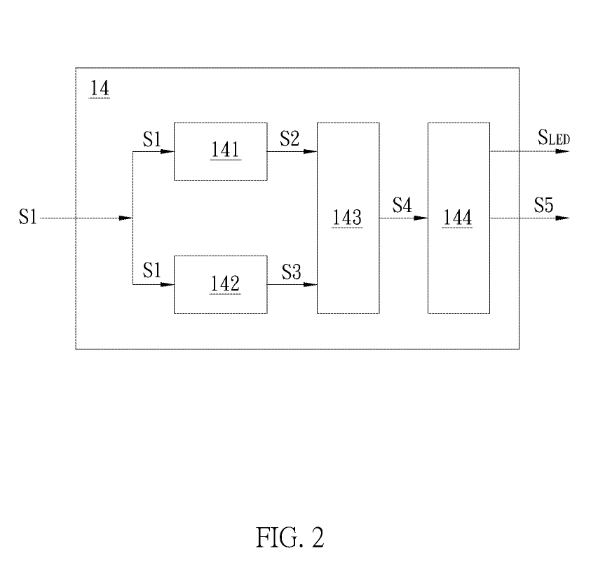

Please refer to FIG. 2. FIG. 2 illustrates a functional block diagram of the first digital processing unit 14. As shown in FIG. 2, the first digital processing unit 14 includes a first calculating unit 141, a second calculating unit 142, a third calculating unit 143 and a fourth calculating unit 144. Wherein, the first calculating unit 141 and the second calculating unit 142 are coupled to the third calculating unit 143; the third calculating unit 143 is coupled to the fourth calculating unit 144. It should be noticed that the circuit structure of the first digital processing unit 14 can be disposed according to practical needs and not limited to this. The first calculating unit 141, the second calculating unit 142, the third calculating unit 143 and the fourth calculating unit 144 of the first digital processing unit 14 all belong to digital calculation logic circuit having adjusting parameters, and their algorithms can be adjusted in response to different usage scenarios.

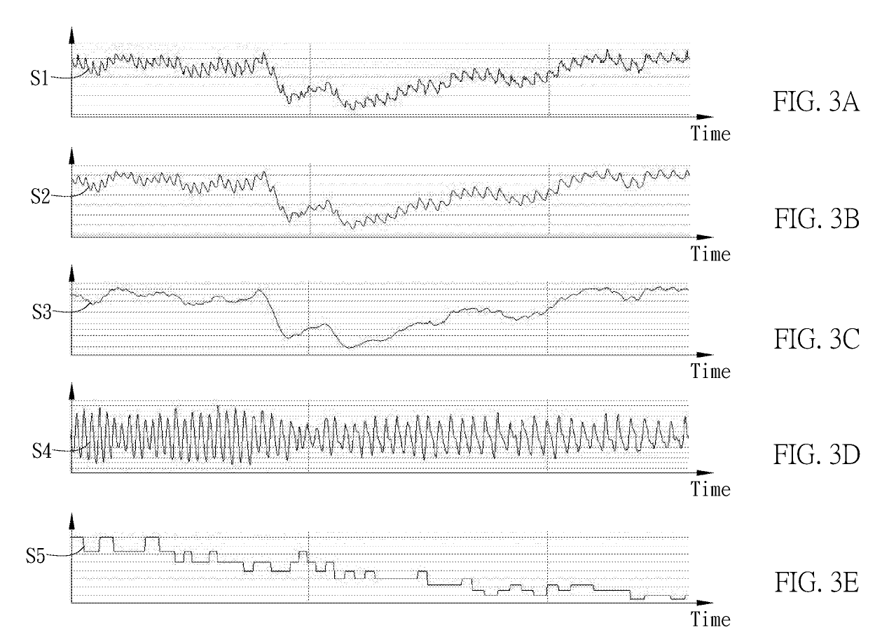

When the first digital processing unit 14 receives the first digital light sensing signal S1 (its waveform diagram please refer to FIG. 3A), the first calculating unit 141 performs a first rolling average calculation on the first digital light sensing signal S1 via a first sample size to obtain the second digital light sensing signal S2 (its waveform diagram please refer to FIG. 3B); the second calculating unit 142 performs a second rolling average calculation on the first digital light sensing signal S1 via a second sample size to obtain the third digital light sensing signal S3 (its waveform diagram please refer to FIG. 3C).

It should be noticed that the first sample size used by the first calculating unit 141 to perform the first rolling average calculation on the first digital light sensing signal S1 is smaller than the second sample size used by the second calculating unit 142 to perform the second rolling average calculation on the first digital light sensing signal S1. In this embodiment, the second sample size can be 3˜10 time of the first sample size, but not limited to this.

In fact, the first calculating unit 141 uses the first sample size having less samples to perform the first rolling average calculation on the first digital light sensing signal S1; that is to say, a low-pass filtering is performed on the first digital light sensing signal S1 to obtain the second digital light sensing signal S2 which is low-pass filtered. The second calculating unit 142 uses the second sample size having more samples to perform the second rolling average calculation on the first digital light sensing signal S1; that is to say, a very low pass filtering is performed on the first digital light sensing signal S1 to obtain the third digital light sensing signal S3 which is very low pass filtered.

Then, the third calculating unit 143 will subtract the third digital light sensing signal S3 from the second digital light sensing signal to remove the high-frequency background noises and the ultra-low frequency moving noises simultaneously and obtain a PPG signal S4 (its waveform diagram please refer to FIG. 3D). As shown in FIG. 3D, the waveform of the PPG signal S4 can be used to represent the pulse variation without the high-frequency background noises and the ultra-low frequency moving noises. Obviously, in this embodiment, the pulse in the beginning has faster frequency, but the pulse becomes slower with the increasing time.

It should be noticed that since the optical sensing apparatus 1 uses the digital calculation method (e.g., the rolling average calculation method) to the PPG signal S4 without the high-frequency background noises and ultra-low frequency moving noises, no high-pass filter including capacitors with high capacitance is necessary in the optical sensing apparatus 1, so that the volume and cost of the optical sensing apparatus 1 can be largely reduced. In addition, since the optical sensing apparatus 1 uses the digital calculation method (e.g., the rolling average calculation method) to average the original signals, it has adaptability to be dynamically adjusted in response to different moving noises.

Then, the fourth calculating unit 144 will detect the times corresponding to every peak values of the waveform of the PPG signal S4 and obtain the heart rate signal S5 (its waveform diagram please refer to FIG. 3E) according to the time interval between two adjacent peak values. As shown in FIG. 3E, the waveform of the heart rate signal S5 can be used to represent the heart rate variation according to the pulse variation. Obviously, in this embodiment, the heart rate in the beginning is faster, but the heart rate becomes slower with the increasing time.

Furthermore, the fourth calculating unit 144 can also determine whether to adjust an output of the light source or adjust the magnification of the sensing signal amplified by the analog processing unit 11 according to the peak value detection result of the waveform of the PPG signal S4. For example, if the fourth calculating unit 144 determines that the output intensity of the light source is insufficient according to the peak value detection result of the waveform of the PPG signal S4, the fourth calculating unit 144 can output a light source adjusting signal S_{LED} to the driving unit 17 and the driving unit 17 will enhance the intensity of the light source through a constant current way, but not limited to this.

The buffer unit 16 is a data temporary storage region used to temporarily store the heart rate signal S5 outputted by the first digital processing unit 14; the transmission interface 18 can be an I2C signal transmission interface used to transmit signals among the analog/digital conversion unit 12, the first digital processing unit 14, the second digital processing unit 15, the buffer unit 16 and the driving unit 17.

(2) When the switching unit 13 switches to the second mode according to the mode control signal MC, the switching unit 13 transmits the first digital light sensing signal 51 to the second digital processing unit 15. The second oscillating unit 20 coupled to the second digital processing unit 15 is a low-speed oscillator used to provide a second clock signal to the second digital processing unit 15.

In this embodiment, the second digital processing unit 15 is a digital signal processor used to generate a proximity sensing signal S6 according to the first digital light sensing signal S1, but not limited to this. The second digital processing unit 15 can obtain the intensity of the reflected light having the specific wavelength received by the sensing unit 10B according to the first digital light sensing signal S1, and then the second digital processing unit 15 can determine a distance from the target according to the intensity of the reflected light. It should be noticed that the operation principle of the second digital processing unit 15 determining the distance from the target according to the intensity of the reflected light is the same with that of the ordinary reflective proximity sensor, but not limited to this.

In practical applications, the optical sensing apparatus 1 can switch to the second mode through the switching unit 13 at first to sense whether the target is near enough the optical sensing apparatus 1. Once the target is near enough the optical sensing apparatus 1, the optical sensing apparatus 1 will switch to the first mode through the switching unit 13 to perform the heart rate sensing on the target to save power consumption of the optical sensing apparatus 1, but not limited to this.

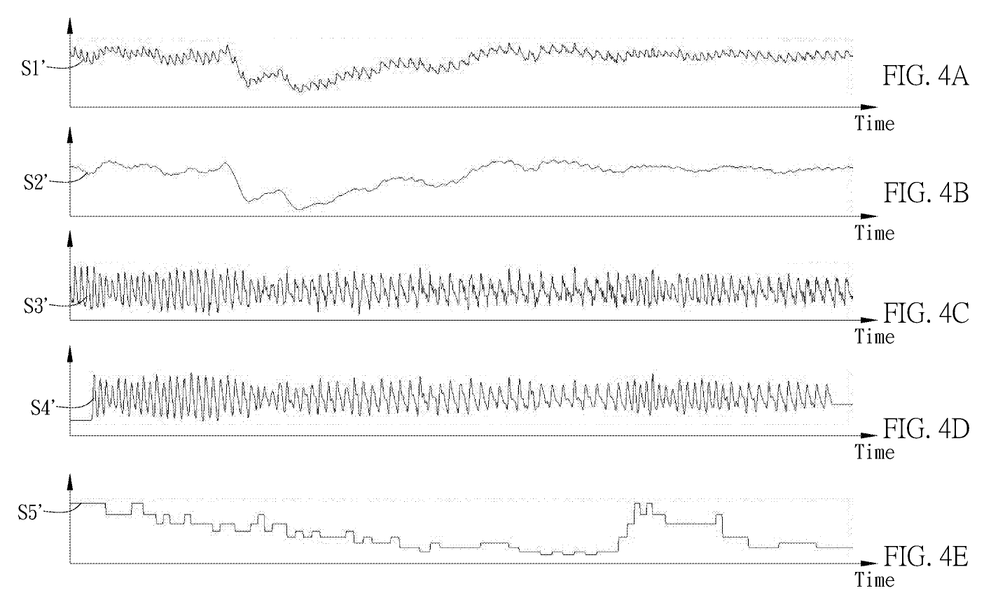

It should be noticed that the practical operation of the first digital processing unit 14 is not limited by the above-mentioned embodiments. In another embodiment, the first digital processing unit 14 can perform the rolling average calculation on the first digital light sensing signal S1′ (its waveform diagram please refer to FIG. 4A) outputted by the analog/digital conversion unit 12 via a large sample size (e.g., 0.5˜1 times of the sampling time) to obtain the second digital light sensing signal S2′ (its waveform diagram please refer to FIG. 4B).

Then, the first digital processing unit 14 subtracts the first digital light sensing signal S1′ from the second digital light sensing signal S2′ to obtain the PPG signal S3′ without the ultra-low frequency moving noises (its waveform diagram please refer to FIG. 4C).

Afterwards, the first digital processing unit 14 performs low-pass filtering on the PPG signal S3′ to further obtain the PPG signal S4′ without the high-frequency background noises (its waveform diagram please refer to FIG. 4D).

At last, the first digital processing unit 14 obtains the heart rate signal S5′ according to the peak values of the PPG signal S4′ (its waveform diagram please refer to FIG. 4E).

Above all, it can be found that the optical sensing apparatus 1 of the invention can effectively remove the high-frequency background noises and ultra-low frequency moving noises from the digital light sensing signal and calculate the heart rate accordingly, and even determine whether to adjust the light source or the magnification of the sensing signal according to the feedback of the calculation result.

Another embodiment of the invention is a method of operating an optical sensing apparatus. In this embodiment, the method is used to operate an optical sensing apparatus having both proximity sensing function and heart rate sensing function, and it can be switched to operate the proximity sensing function or the heart rate sensing function according to practical needs. Since it is small and cheap, it can be widely used in smart phones, tablet PCs, notebook PCs and various kinds of wearable electronic devices, but not limited to this.

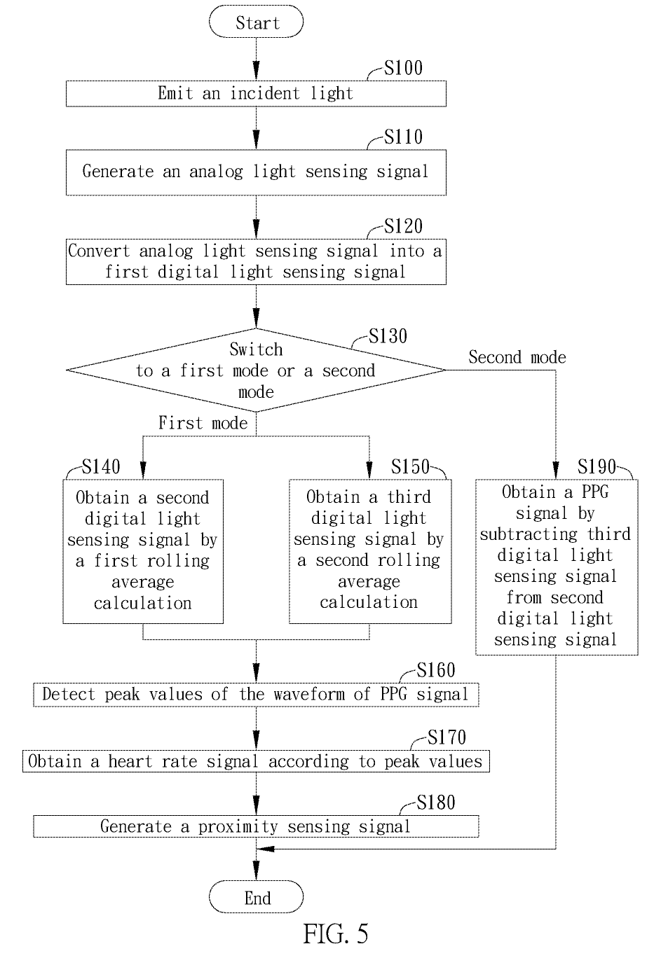

Please refer to FIG. 5. FIG. 5 illustrates a flowchart of the optical sensing apparatus operating method in this embodiment. As shown in FIG. 5, in the step S100, the method controls the light source unit to emit an incident light having a specific wavelength. When the light source unit emits the incident light having the specific wavelength to a target and reflected by the target, in the step S110, the method receives a reflected light having the specific wavelength and performs analog processing procedures on the reflected light to output an analog light sensing signal.

In the step S120, the method converts the analog light sensing signal into a digital light sensing signal. In the step S130, the method selectively switches the optical sensing apparatus to a first mode or a second mode according to a mode control signal.

When the optical sensing apparatus is switched to the first mode, in the steps S140 and S150, the method performs a first rolling average calculation on the first digital light sensing signal via a first sample size to obtain a second digital light sensing signal and performs a second rolling average calculation on the first digital light sensing signal via a second sample size to obtain a third digital light sensing signal.

It should be noticed that the first sample size used to perform the first rolling average calculation on the first digital light sensing signal is smaller than the second sample size used to perform the second rolling average calculation on the first digital light sensing signal. In this embodiment, the first sample size can be 0.1˜0.2 times of a sampling time and the second sample size can be 0.5-1 times of the sampling time, but not limited to this.

Then, in the step S160, the method subtracts the third digital light sensing signal from the second digital light sensing signal to remove the high-frequency background noises and ultra-low frequency moving noises and obtain a PPG signal.

Afterwards, in the step S170, the method detects the times corresponding to every peak values of the waveform of the PPG signal. At last, in the step S180, the method obtains a heart rate signal according to a time interval between two adjacent peak values.

On the other hand, when the optical sensing apparatus is switched to the second mode, in the step S190, the method generates a proximity sensing signal according to the digital light sensing signal.

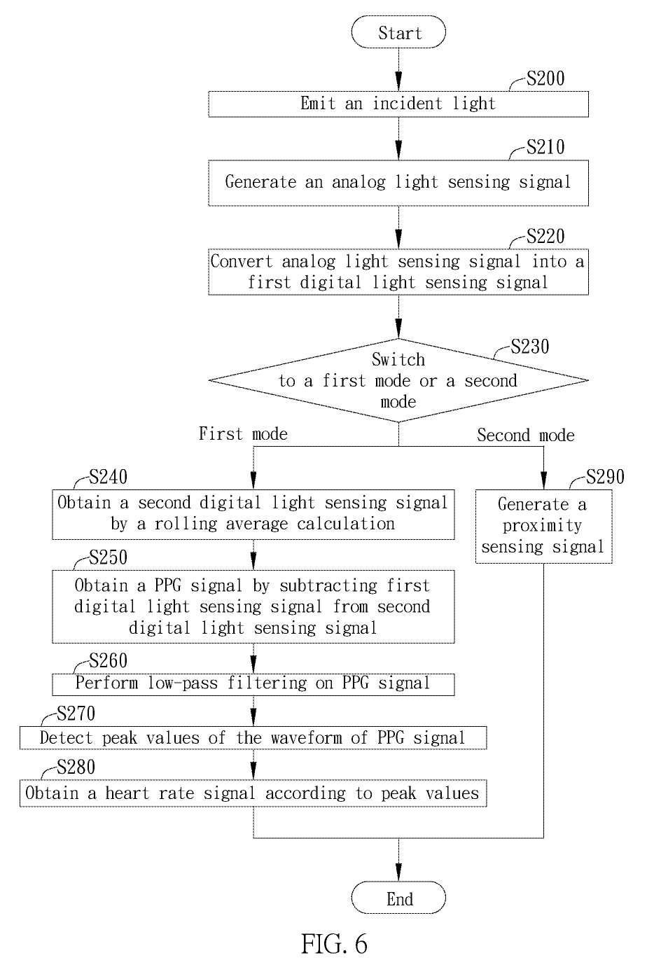

Please refer to FIG. 6. FIG. 6 illustrates a flowchart of the optical sensing apparatus operating method in another embodiment. As shown in FIG. 6, in the step S200, the method controls the light source unit to emit an incident light having a specific wavelength. When the light source unit emits the incident light having the specific wavelength to a target and reflected by the target, in the step S210, the method receives a reflected light having the specific wavelength and performs analog processing procedures on the reflected light to output an analog light sensing signal.

In the step S220, the method converts the analog light sensing signal into a digital light sensing signal. In the step S230, the method selectively switches the optical sensing apparatus to a first mode or a second mode according to a mode control signal.

When the optical sensing apparatus is switched to the first mode, in the step S240, the method performs a rolling average calculation on the first digital light sensing signal via a sample size to obtain a second digital light sensing signal. Then, in the step S250, the method subtracts the first digital light sensing signal from the second digital light sensing signal to remove the ultra-low frequency moving noises and obtain a PPG signal.

It should be noticed that the sample size used to perform the rolling average calculation on the first digital light sensing signal can be 0.5˜1 times of the sampling time, but not limited to this.

Then, in the step S260, the method performs low-pass filtering on the PPG signal to further remove the high-frequency background noises from the PPG signal.

Afterwards, in the step S270, the method detects the times corresponding to every peak values of the waveform of the PPG signal. At last, in the step S280, the method obtains a heart rate signal according to a time interval between two adjacent peak values.

On the other hand, when the optical sensing apparatus is switched to the second mode, in the step S290, the method generates a proximity sensing signal according to the digital light sensing signal.

Compared to the prior arts, the optical sensing apparatus and operating method thereof in the invention will amplify the analog sensing signal of the optical sensor and convert it into the digital sensing signal through high-resolution analog/digital conversion, and then use digital way to reduce the background noises and moving noises and calculate the heart rate, and feedback the calculating result to determine whether to adjust the light source or the magnification of the sensing signal. The optical sensing apparatus and operating method thereof in the invention have following advantages:

(1) The optical sensing apparatus of the invention can have both the proximity sensing function and heart rate sensing function, and the light source and the corresponding optical sensor are disposed at the same side of the optical sensing apparatus, when the optical sensing apparatus contacts with the human body, the intensity of the emitting light can be adjusted to penetrate through the skin and enter into the artery, and then the optical sensor senses the intensity of the reflected lights and performs detection and calculation of the PPG signal in the reflected light sensor mode.

(2) The optical sensing apparatus of the invention uses the digital ways (the rolling average calculation method and low-pass filtering method) to remove the high-frequency background noises and ultra-low frequency moving noises from the digital light sensing signal; therefore, no high-pass filter including capacitors with high capacitance is necessary in the optical sensing apparatus to largely reduce the volume and cost of the optical sensing apparatus. In addition, since the optical sensing apparatus of the invention uses the digital calculation method (the rolling average calculation method) to average the original signals, it has adaptability to be dynamically adjusted in response to different moving noises.

(3) The optical sensing apparatus of the invention uses the analog/digital converter having high resolution to convert the analog light sensing signal into the digital light sensing signal to retain a high dynamic range in response to different background values under different usage scenarios. In addition, since the high-frequenct background noises and ultra-low frequency moving noises are removed from the digital light sensing signal, the optical sensing apparatus of the invention only needs simple calculation structure to obtain the peak values of the PPG signal to calculate the heart rate, the circuit structure of the optical sensing apparatus of the invention can be simplified and the cost can be decreased.

(4) The optical sensing apparatus of the invention can be switched to operate the proximity sensing function or the heart rate sensing function according to practical needs. Since it is small and cheap, it can be widely used in smart phones, tablet PCs, notebook PCs and various kinds of wearable electronic devices and have great market potential.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. An optical sensing apparatus, comprising:
a sensing unit configured to provide a light sensing signal;
an analog/digital conversion unit coupled to the sensing unit and configured to convert the light sensing signal into a digital light sensing signal;
a switching unit coupled to the analog/digital conversion unit and configured to receive a mode control signal and selectively switch to a first mode or a second mode;
a first processing unit coupled to the analog/digital conversion unit and the switching unit; and
a second processing unit coupled to the analog/digital conversion unit and the switching unit,
wherein when the switching unit switches to the first mode according to the mode control signal, the first processing unit generates a photoplethysmography (PPG) signal according to the digital light sensing signal; when the switching unit switches to the second mode according to the mode control signal, the second processing unit generates a proximity sensing signal according to the digital light sensing signal.

2. The optical sensing apparatus of claim 1, wherein the first processing unit performs a first rolling average calculation and a second rolling average calculation on the digital light sensing signal via a first sample size and a second sample size respectively, and the second sample size is larger than the first sample size.

3. The optical sensing apparatus of claim 2, wherein the first sample size is 0.1˜0.2 times of a sampling time and the second sample size is 0.5˜1 times of the sampling time.

4. The optical sensing apparatus of claim 2, wherein after the first processing unit further obtains the digital light sensing signal without high-frequency noises and ultra-low frequency motion noises according to calculation results of the first rolling average calculation and the second rolling average calculation, the first processing unit detects a PPG peak value and generates the PPG signal according to a detection result of the PPG peak value.

5. The optical sensing apparatus of claim 4, wherein the first processing unit further determines whether to adjust a light source output or adjust a magnification of the light sensing signal according to the detection result of the PPG peak value.

6. The optical sensing apparatus of claim 1, wherein the first processing unit performs a rolling average calculation on the digital light sensing signal via a sample size and removes ultra-low frequency motion noises from the digital light sensing signal according to a calculation result of the rolling average calculation and removes high-frequency noises from the digital light sensing signal through low-pass filtering, and then the first processing unit detects a PPG peak value and generates the PPG signal according to a detection result of the PPG peak value.

7. The optical sensing apparatus of claim 6, wherein the sample size is 0.1˜0.2 times of a sampling time.

8. The optical sensing apparatus of claim 1, wherein the mode control signal is generated by an application program performed by the optical sensing apparatus.

9. The optical sensing apparatus of claim 1, wherein the mode control signal is generated when the optical sensing apparatus is continuously pressed for a default time.

10. A method of operating an optical sensing apparatus, comprising:
(a) the optical sensing apparatus converting the light sensing signal into a digital light sensing signal;
(b) the optical sensing apparatus selectively switching to a first mode or a second mode according to a mode control signal;
(c1) when the optical sensing apparatus switches to the first mode, the optical sensing apparatus generating a photoplethysmography (PPG) signal according to the digital light sensing signal; and
(c2) when the optical sensing apparatus switches to the second mode, the optical sensing apparatus generating a proximity sensing signal according to the digital light sensing signal.

11. The method of claim 10, wherein the step (c1) performs a first rolling average calculation and a second rolling average calculation on the digital light sensing signal via a first sample size and a second sample size respectively, and the second sample size is larger than the first sample size.

12. The method of claim 11, wherein the first sample size is 0.1˜0.2 times of a sampling time and the second sample size is 0.5˜1 times of the sampling time.

13. The method of claim 11, wherein the step (c1) further obtains the digital light sensing signal without high-frequency noises and ultra-low frequency motion noises according to calculation results of the first rolling average calculation and the second rolling average calculation, and then detects a PPG peak value and generates the PPG signal according to a detection result of the PPG peak value.

14. The method of claim 13, further comprising:
determining whether to adjust a light source output or adjust a magnification of the light sensing signal according to the detection result of the PPG peak value.

15. The method of claim 10, wherein the step (c1) performs a rolling average calculation on the digital light sensing signal via a sample size and removes ultra-low frequency motion noises from the digital light sensing signal according to a calculation result of the rolling average calculation and removes high-frequency noises from the digital light sensing signal through low-pass filtering, and then detects a PPG peak value and generates the PPG signal according to a detection result of the PPG peak value.

16. The method of claim 15, wherein the sample size is 0.1˜0.2 times of a sampling time.

17. The method of claim 10, wherein the mode control signal is generated by an application program performed by the optical sensing apparatus.

18. The method of claim 10, wherein the mode control signal is generated when the optical sensing apparatus is continuously pressed for a default time.
